(19) United States
(12) Patent Application Publication
(10) Pub. No.: US 2024/0350013 A1
(43) Pub. Date: Oct. 24, 2024

(54) WEARABLE SENSOR

(71) Applicant: Sharp Display Technology Corporation, Kameyama City (JP)

(72) Inventors: Takeshi HARA, Kameyama City (JP); Hisao UEMOTO, Kameyama City (JP); Mitsuru CHIDA, Kameyama City (JP); Midori KAGEYAMA, Kameyama City (JP); Rikiya TAKITA, Kameyama City (JP); Yang QU, Kameyama City (JP)

(21) Appl. No.: 18/589,741

(22) Filed: Feb. 28, 2024

(30) Foreign Application Priority Data
Apr. 21, 2023 (JP) 2023-070193

(51) Int. Cl. A61B 5/00

(52) U.S. Cl. CPC A61B 5/0059; A61B 2562/0238; A61B 2562/164

(57) ABSTRACT

Provided is a wearable sensor with enhanced light sensitivity. The wearable sensor is to be worn by a subject, the wearable sensor including: a supporting substrate; and sensor elements placed on the supporting substrate. The sensor elements each include: a light emitter which is an organic light emitting diode or an inorganic light emitting diode; and a reflected light receiver configured to measure a quantity of light emitted from the light emitter and reflected in a living body of a subject. Preferably, in a plan view, at least part of the reflected light receiver does not overlap the light emitter.

### CROSS-REFERENCE TO RELATED APPLICATIONS

The present application claims priority under 35 U.S.C. § 119 to Japanese Patent Application No. 2023-070193 filed on Apr. 21, 2023, the contents of which are incorporated herein by reference in their entirety.

### BACKGROUND OF THE INVENTION

### Field of the Invention

The following disclosure relates to wearable sensors.

### Description of Related Art

With the increase in health consciousness, wearable sensors that can easily measure information on one's physical condition are attracting attention from the viewpoints of lifestyle improvement, early detection of diseases, and health management.

For example, JP 2019-506205 T discloses a monitoring device adapted to be removably attachable to a subject's body, the device comprising: a measuring unit, comprising: at least two light emitting sources; and at least one sensor, to detect light beams emitted from the at least two light emitting sources; and a controller, coupled to the measuring unit, and configured to measure and analyze physiological signs of the subject.

JP 6642421 B discloses a measurement device, comprising: a light source configured to emit light having a predetermined wavelength; a polarizer configured to convert the light emitted from the light source to linearly polarized light; a modulator configured to modulate a polarization direction of the linearly polarized light; mirrors each individually arranged on a side of a polygon partially open along a periphery of a measured object, which is a living body, and configured to totally reflect the light modulated in the modulator toward the measured object to cause the light to pass through the measured object along the periphery of the measured object; an analyzer configured to separate, on the basis of a polarization direction of transmission light transmitted through the measured object, scattered light scattered in the measured object from the transmission light; and a detector configured to detect the transmission light separated from the scattered light in the analyzer.

### BRIEF SUMMARY OF THE INVENTION

FIG. 9 is a schematic cross-sectional view of the measuring unit disclosed in JP 2019-506205 T. FIG. 10 is a schematic cross-sectional view showing reflection of light in a living body. The directions of the dashed line arrows in FIG. 9 and FIG. 10 each indicate a direction of a light beam. For example, a measuring unit 300R in JP 2019-506205 T shown in FIG. 9 is placed adjacent to skin 30 of a subject. A light beam is then emitted from a light emitting source 320R and into the skin 30 such that it penetrates the skin 30 and reflected back from a blood vessel 31 towards a sensor 310R. The difference in the data between the emitted beam and the received (reflected) beam provides an indication on the radiation absorption by the blood in the blood vessel 31 and thus indicates characteristics and blood measurements of the blood inside the blood vessel 31, in a non-invasive procedure. The measuring unit 300R further includes an ultrasonic unit 340R capable of transmitting and receiving ultrasound signals.

Biosensors using laser light or light emitting diode (LED) light as with the measuring unit disclosed in JP 2019-506205 T shown in FIG. 9 have already been put to practical use. However, as light emitting diodes each are several millimeter-sized, space allocation for the light emitting diodes has been an issue for conventional biosensors. This means that conventional biosensors, unsuitable for installation of many light emitting light sources and many light receivers, include only a few light sources and a few light receivers to end up with insufficient light sensitivity. Also, since light reflected in a living body scatters as shown in FIG. 10, conventional biosensors would receive only faint light to fail to achieve sufficient light sensitivity.

JP 2019-506205 T and JP 6642421 B do not disclose a wearable sensor with enhanced light sensitivity. In response to the above issues, an object of the present invention is to provide a wearable sensor with enhanced light sensitivity.
(1) One embodiment of the present invention is directed to a wearable sensor to be worn by a subject, the wearable sensor including: a supporting substrate; and sensor elements placed on the supporting substrate, the sensor elements each including: a light emitter which is an organic light emitting diode or an inorganic light emitting diode; and a reflected light receiver configured to measure a quantity of light emitted from the light emitter and reflected in a living body of a subject.
(2) In an embodiment of the present invention, the wearable sensor includes the structure (1), and in a plan view, at least part of the reflected light receiver does not overlap the light emitter.
(3) In an embodiment of the present invention, the wearable sensor includes the structure (1) or (2), the reflected light receiver includes, sequentially from a supporting substrate side, a switching element for reflected light reception and a sensor unit for reflected light reception, and the sensor unit for reflected light reception includes, sequentially from the supporting substrate side, a bottom electrode for reflected light reception, a photoelectric conversion layer for reflected light reception, and a top electrode for reflected light reception.
(4) In an embodiment of the present invention, the wearable sensor includes the structure (3), and the switching element for reflected light reception includes a semiconductor layer for reflected light reception which contains at least one of amorphous silicon, low-temperature polysilicon, or indium gallium zinc oxide.
(5) In an embodiment of the present invention, the wearable sensor includes the structure (3) or (4), and the switching element for reflected light reception has a double-gate structure.
(6) In an embodiment of the present invention, the wearable sensor includes the structure (1), (2), (3), (4), or (5), and further includes an emitted light receiver configured to measure a quantity of light emitted from the light emitter and not passing through a living body of a subject.
(7) In an embodiment of the present invention, the wearable sensor includes the structure (6), the emitted light receiver includes, sequentially from a supporting substrate side, a switching element for emitted light reception and a sensor unit for emitted light reception, and the sensor unit for emitted light reception includes, sequentially from the supporting substrate side, a bottom electrode for emitted light reception, a photoelectric conversion layer for emitted light reception, and a top electrode for emitted light reception.
(8) In an embodiment of the present invention, the wearable sensor includes the structure (7), and the switching element for emitted light reception includes a semiconductor layer for emitted light reception which contains at least one of amorphous silicon, low-temperature polysilicon, or indium gallium zinc oxide.
(9) In an embodiment of the present invention, the wearable sensor includes the structure (7) or (8), and the switching element for emitted light reception has a double-gate structure.
(10) In an embodiment of the present invention, the wearable sensor includes the structure (6), (7), (8), or (9), the light emitter includes, sequentially from a supporting substrate side, a reflection electrode and a light emitting layer, the emitted light receiver includes a photoelectric conversion layer for emitted light reception, the wearable sensor includes, sequentially from the supporting substrate side, the photoelectric conversion layer for emitted light reception, the reflection electrode, and the light emitting layer, and in a plan view, at least part of the light emitting layer overlaps the photoelectric conversion layer for emitted light reception without the reflection electrode in between.
(11) In an embodiment of the present invention, the wearable sensor includes the structure (6), (7), (8), (9), or (10), the light emitter includes, sequentially from a supporting substrate side, a reflection electrode and a light emitting layer, the emitted light receiver includes a photoelectric conversion layer for emitted light reception, the wearable sensor comprises, sequentially from the supporting substrate side, the photoelectric conversion layer for emitted light reception, the reflection electrode, and the light emitting layer, and in a plan view, at least part of a region of the reflection electrode overlapping the photoelectric conversion layer for emitted light reception is provided with an aperture.
(12) In an embodiment of the present invention, the wearable sensor includes the structure (1), (2), (3), (4), (5), (6), (7), (8), (9), (10), or (11), the light emitter includes, sequentially from the supporting substrate side, a reflection electrode and a light emitting layer, and in a plan view, at least part of the light emitting layer overlaps the reflection electrode.
(13) In an embodiment of the present invention, the wearable sensor includes the structure (1), (2), (3), (4), (5), (6), (7), (8), (9), (10), (11), or (12), and the supporting substrate is a flexible substrate.

The present invention can provide a wearable sensor with enhanced light sensitivity.

### BRIEF DESCRIPTION OF THE DRAWINGS

FIG. 1 is a schematic perspective view of a wearable sensor of Embodiment 1.

FIG. 2 is a schematic view showing an example of a function of the wearable sensor of Embodiment 1.

FIG. 3 is a schematic cross-sectional view of the wearable sensor of Embodiment 1.

FIG. 4 is an example of a circuit diagram of a first light receiver in the wearable sensor of Embodiment 1.

FIG. 5 is a schematic perspective view of a wearable sensor of Embodiment 2.

FIG. 6 is a schematic view showing an example of a function of the wearable sensor of Embodiment 2.

FIG. 7 is a schematic cross-sectional view of the wearable sensor of Embodiment 2.

FIG. 8 is an example of a circuit diagram of a second light receiver in the wearable sensor of Embodiment 2.

FIG. 9 is a schematic cross-sectional view of the measuring unit disclosed in JP 2019-506205 T.

FIG. 10 is a schematic cross-sectional view showing reflection of light in a living body.

### DETAILED DESCRIPTION OF THE INVENTION

Hereinafter, embodiments of the present invention are described. The present invention is not limited to the contents of the following embodiments. The design may be modified as appropriate within the range satisfying the configuration of the present invention. In the following description, components having the same or similar functions in different drawings are commonly provided with the same reference sign so as to appropriately avoid repetition of description. The structures in the present invention may be combined as appropriate without departing from the gist of the present invention.

### Embodiment 1

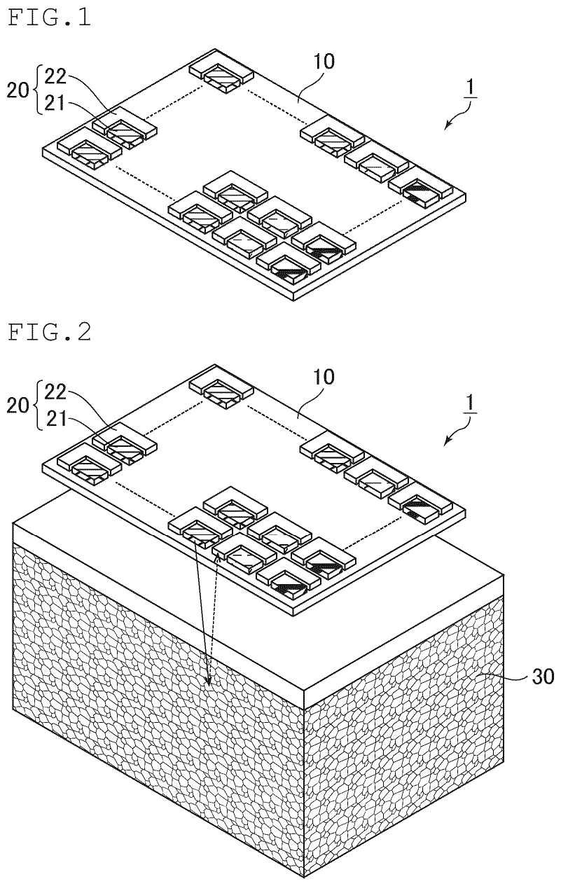

FIG. 1 is a schematic perspective view of a wearable sensor of Embodiment 1. A wearable sensor 1 of the present embodiment is a wearable sensor to be worn by a subject and includes a supporting substrate 10 and sensor elements 20 placed on the supporting substrate 10. The sensor elements 20 each include a light emitter 21 which is an organic light emitting diode (OLED) or an inorganic light emitting diode (inorganic LED); and a reflected light receiver 22 configured to measure the quantity of light emitted from the light emitter 21 and reflected in the living body of a subject. In this configuration, the space to which each light emitter 21 is allocated can be reduced, so that more sensor elements 20 can be arranged in arrays in the wearable sensor 1. As a result, the light sensitivity can be enhanced. Also, since a flexible substrate (also referred to as a flexible base material) is used as the supporting substrate 10, the wearability of the sensor can be improved.

The wearable sensor 1 of the present embodiment specifically includes arrays of light emitting elements using an OLED display technology which correspond to the light emitters 21 and of semiconductor sensors using an X-ray sensor technology which correspond to the reflected light receivers 22 to enable a biosensor and thus solve the problem. Including high-definition light emitting elements and light receiving elements arranged in arrays having a certain area, the wearable sensor 1 can efficiently receive light reflected in a living body (including light scattered in the living body). The wearable sensor 1 is also less susceptible to the influence of differences in blood vessel distributions among individuals, thus having higher accuracy and higher sensitivity as a biosensor. In addition, including light emitting elements and semiconductor sensors formed on a flexible substrate, the wearable sensor 1 can be a device with improved wearability.

As described above, the wearable sensor 1 of the present embodiment uses the OLED display technology and the X-ray sensor technology to arrange light emitting sources and detection sensors in arrays having a certain area with high precision. The wearable sensor 1 thus can receive light reflected in a living body efficiently, demonstrating enhanced sensitivity. Including the sensor elements 20 formed on the flexible substrate, the wearable sensor 1 can be a device with high wearability.

JP 2019-506205 T discloses a biosensor that can be placed on a human body. The biosensor disclosed in JP 2019-506205 T including at least two light emitting sources and at least one detection sensor can measure and analyze physiological signs of a human body. The wavelength of emitted light is a specific wavelength between 400 to 2500 nm. Data analysis allows the conditions of diabetes, dehydration, and medications to be monitored.

The biosensor disclosed in JP 2019-506205 T, in which only a few light emitting sources and a few detection sensors can be placed as shown in FIG. 9, has low sensitivity as it cannot efficiently receive light reflected in a living body. In addition, this biosensor has poor wearability as it is a rigid device.

JP 6642421 B discloses a sensor including a light source configured to emit light having a predetermined wavelength; a polarizer configured to convert the light emitted from the light source to linearly polarized light; mirrors configured to reflect the light within the measured object; and a detector. The sensor disclosed in JP 6642421 B is devised to show high biosensing accuracy by repeated reflection. Although requiring strong light sources and mirrors to repeat reflection many times, the sensor disclosed in JP 6642421 B cannot include therein multiple light sources and mirrors. Thus, the sensor cannot efficiently receive scattered light. The reflection repeated many times also causes more scattering of light. In addition, the sensor has poor wearability as it is a rigid device.

Hereinafter, the wearable sensor 1 of the present embodiment is described in detail.

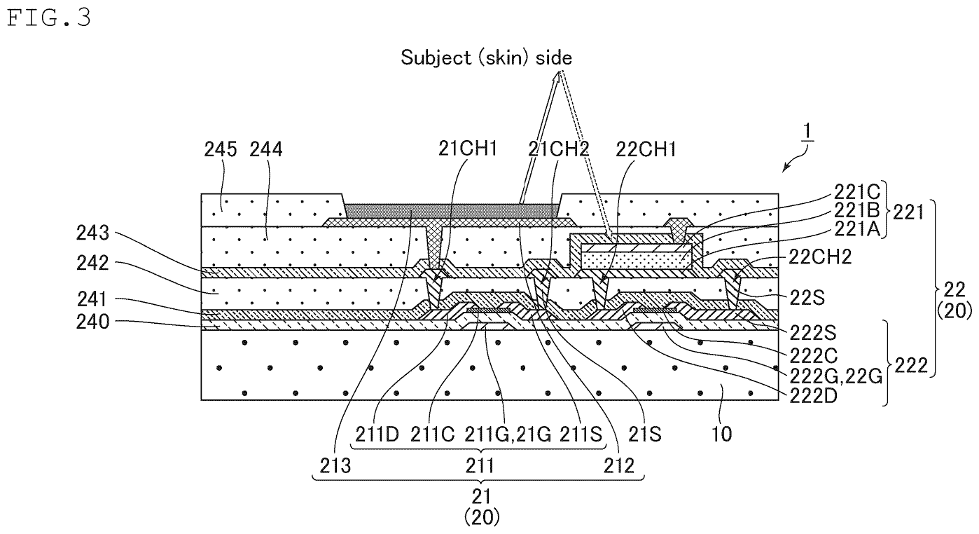

FIG. 2 is a schematic view showing an example of a function of the wearable sensor of Embodiment 1. FIG. 3 is a schematic cross-sectional view of the wearable sensor of Embodiment 1. In FIG. 2 and FIG. 3, the solid line arrow indicates emission light from a light emitter, and the dashed line arrow indicates light reflected in the living body. As shown in FIG. 2 and FIG. 3, the wearable sensor 1 of the present embodiment is, for example, a wearable sensor to be worn on the skin 30 of a human or animal to measure the state in the living body of the subject.

The wearable sensor 1 includes the sensor elements 20. The sensor elements (also referred to as pixels) 20 each include a light emitter 21 which is an OLED or inorganic LED and a reflected light receiver 22 that measures the quantity of light emitted from the light emitter 21 and reflected in the living body of a subject. The sensor elements 20 may be arranged in arrays on the supporting substrate 10.

The supporting substrate 10 may be any substrate that supports the sensor elements 20. Examples of the supporting substrate 10 include flexible substrates and glass substrates. The supporting substrate 10 is preferably a flexible substrate. This configuration increases flexibility of the wearable sensor 1 to improve the wearability of the wearable sensor 1. The flexible substrate means a film substrate that is made of a synthetic resin material (e.g., polyimide-based resin) and is insulative and bendable without breaking.

The supporting substrate 10 may be placed on or near the side coming into contact with the subject or may be placed on or near the side opposite to the side coming into contact with the subject. When the supporting substrate 10 is placed on or near the side coming into contact with the subject, the supporting substrate 10 is preferably transparent to light. When the supporting substrate 10 is placed on or near the side coming into contact with the subject, the transmittance of the supporting substrate 10 is, for example, 80% or higher and 100% or lower, preferably 90% or higher and 100% or lower, more preferably 95% or higher and 100% or lower. The transmittance can be measured by the measurement method in conformity with JIS-K-7375.

The sensor elements 20 are arranged, for example, in one direction (e.g., “row direction”) and in a direction crossing the one direction (e.g., “column direction”) to form a matrix pattern. The wearable sensor 1 includes multiple sensor elements 20. This configuration can enhance the light sensitivity.

Each of the light emitters 21 is an OLED or an inorganic LED. The light emitter 21 may be, for example, a light emitting element using an OLED display technology. The light emitter 21 may include a switching element for light emission which has a function of controlling emission of light.

The light emitter 21 preferably emits light having a wavelength suitable for sensing of a living body. For example, near-infrared light, which can deeply penetrate into a living body, can increase the amount of living body information to be obtained. The light emitter 21, for example, preferably emits light with a wavelength of 700 nm or longer and 2500 nm or shorter, more preferably a wavelength of 700 nm or longer and 1700 nm or shorter, still more preferably a wavelength of 1000 nm or longer and 1700 nm or shorter. The light emitter 21 may emit visible light (e.g., light with a wavelength of 380 nm or longer and shorter than 700 nm). This configuration allows living body information to be obtained through sensing with visible light.

The light emitter 21 preferably emits lights with different wavelengths. This configuration can increase the amount of living body information to be obtained. The light emitter 21 preferably changes the quantity of light to be emitted. This configuration allows sensing with different quantities of light, thus increasing the amount of living body information to be obtained.

As shown in FIG. 3, the wearable sensor 1 includes, on the supporting substrate 10, gate lines 21G for light emission parallel to one another and source lines 21S for light emission parallel to one another and extending in a direction crossing the gate lines 21G for light emission. The gate lines 21G for light emission and the source lines 21S for light emission as a whole are formed in a grid pattern to partition the sensor elements 20. Switching elements 211 for light emission are placed at or near the intersections of the gate lines 21G for light emission and the source lines 21S for light emission.

Each light emitter 21 includes, sequentially from the supporting substrate 10 side, a switching element 211 for light emission, a reflection electrode 212, and a light emitting layer 213. Specifically, the light emitter 21 includes, sequentially from the supporting substrate 10 side, a switching element 211 for light emission, a first insulating film 241, a second insulating film 242, a third insulating film 243, a fourth insulating film 244, a reflection electrode 212, and a light emitting layer 213. The switching element 211 for light emission includes, sequentially from the supporting substrate 10 side, a gate electrode 211G for light emission, a gate insulating film 240, a semiconductor layer 211C for light emission, a drain electrode 211D for light emission, and a source electrode 211S for light emission.

The gate insulating film 240, the first insulating film 241, the second insulating film 242, the third insulating film 243, the fourth insulating film 244, and the later-described fifth insulating film 245 are each an inorganic insulating film, an organic insulating film, or a laminate of an organic insulating film and an inorganic insulating film.

Examples of the inorganic insulating film include an inorganic film (relative dielectric constant ε=5 to 7) made of silicon nitride (SiNx) or silicon oxide (SiO₂) and laminates of such films. The thickness of the inorganic insulating film is, for example, 1500 Å or more and 3500 Å or less. Examples of the organic insulating film include organic films with a low relative dielectric constant (relative dielectric constant ε=2 to 5) such as photosensitive acrylic resins and laminates of such films. The thickness of the organic insulating film is not limited and may be, for example, 2 μm or more and 4 μm or less.

The gate insulating film 240, the first insulating film 241, and the third insulating film 243 are each preferably an inorganic insulating film made of SiN, SiO, or SiON, for example. The second insulating film 242, the fourth insulating film 244, and the fifth insulating film are each preferably an acrylic resin-based organic insulating film.

The switching element 211 for light emission has a function of controlling light emission from the light emitting layer 213. The switching element 211 for light emission is a three-terminal switch including a gate electrode 211G for light emission, a source electrode 211S for light emission, a drain electrode 211D for light emission, and a semiconductor layer 211C for light emission. Examples of the switching element 211 for light emission include thin film transistors (TFTs). The switching element 211 for light emission preferably has a double-gate structure.

The “gate electrode” herein is one of the three electrodes constituting a TFT (the rest are a source electrode and a drain electrode) and modulates the amount of electric charge induced to the channel region of the semiconductor layer according to the voltage applied to the gate electrode to control the current flowing between the corresponding source and drain electrodes. The “source electrode” herein is one of the three electrodes constituting a TFT and functions as a source of supply of carriers flowing in the semiconductor layer of the TFT. The “drain electrode” herein is one of the three electrodes constituting a TFT and functions as a source of supply of carriers flowing in the semiconductor layer of the TFT.

The “semiconductor layer” herein encompasses not only layers having the characteristics of a semiconductor (e.g., channel region) but also layers (e.g., source region and drain region) obtained through resistance reduction treatment on layers having the characteristics of a semiconductor such that their specific resistance is lower than that of a channel region. Examples of the material of the semiconductor layer include amorphous silicon (a-Si), low-temperature polysilicon (LTPS), indium gallium zinc oxide (IGZO), and LTPS+IGZO (TOPS).

The semiconductor layer 211C for light emission included in the switching element 211 for light emission preferably contains at least one of LTPS or IGZO. In other words, the semiconductor layer 211C for light emission included in the switching element 211 for light emission preferably contains LTPS or IGZO. In this configuration, sufficient charges and currents can be supplied to the light emitting layer. The semiconductor layer 211C for light emission included in the switching element 211 for light emission more preferably contains LTPS with high electron mobility or IGZO with high electron mobility.

The drain electrode 211D for light emission is connected to the reflection electrode 212 via a contact hole 21CH1. The source electrode 211S for light emission is connected to the corresponding source line for light emission via a contact hole 21CH2.

The reflection electrode 212 contains a metal material with a high reflectance. The reflection electrode 212 contains, for example, aluminum, an aluminum alloy, silver, or a silver alloy. Light emitted from the light emitting layer 213 is reflected by the reflection electrode 212 to then be emitted toward the subject.

The light emitting layer 213 contains an organic luminous material or an inorganic luminous material. The light emitting layer 213 may contain a luminous material including nanoparticles or may have a structure that functions as a light emitting element obtained by dividing a LED element fabricated on a Si wafer and arranging the divided pieces by a pick-and-place method.

When the light emitter 21 is an OLED, the light emitting layer 213 contains an organic luminous material. A light emitting layer 213 containing an organic luminous material is also referred to as an organic light emitting layer. An OLED can be formed by deposition or coating. An OLED can contain an additional organic material for efficient electron transport when appropriate in addition to the luminous substance.

The OLED includes at least one organic light emitting layer between two electrodes. For example, the OLED can include, between the organic light emitting layer and an electrode, an electron injection or transport layer such as an electron transport layer, an electron injection layer, a hole transport layer, a hole injection layer, a hole blocking layer, or an electron blocking layer. Two or more OLED light emitting units each including at least one organic light emitting layer between two electrodes may be laminated as appropriate.

The OLED light emitting unit means the minimum unit including an organic light emitting layer and capable of emitting light during voltage application. An intermediate conductive layer or a charge generating layer is favorably placed between the OLED light emitting units. A conductive material usable as an electrode material is favorable in forming an intermediate conductive layer. The at least two OLED light emitting units laminated may be designed to emit lights of the same color or may be designed to emit lights of different colors. For example, in an OLED having a laminated structure, the at least two OLED light emitting units are favorably designed to emit lights of different colors to cause the OLED to emit white light.

When the light emitter 21 is an inorganic LED, the light emitting layer 213 contains an inorganic luminous material. The light emitting layer 213 containing an inorganic luminous material is also referred to as an inorganic light emitting layer. The inorganic LED can be formed by deposition or coating, or by fabricating an inorganic LED element on a Si wafer and arranging the inorganic LED element by a pick-and-place method. An inorganic LED formed by a pick-and-place method is also referred to as a micro-LED. An inorganic LED can contain an additional inorganic material for efficient electron transport when appropriate in addition to the luminous substance.

The inorganic LED includes at least one inorganic light emitting layer between two electrodes. For example, the inorganic LED can include, between the inorganic light emitting layer and an electrode, an electron injection or transport layer such as an electron transport layer, an electron injection layer, a hole transport layer, a hole injection layer, a hole blocking layer, or an electron blocking layer. Two or more inorganic LED light emitting units each including at least one inorganic light emitting layer between two electrodes may be laminated as appropriate.

The inorganic LED light emitting unit means the minimum unit including an inorganic light emitting layer and capable of emitting light during voltage application. An intermediate conductive layer or a charge generating layer is favorably placed between the inorganic LED light emitting units. A conductive material usable as an electrode material is favorable in forming an intermediate conductive layer. The at least two inorganic LED light emitting units laminated may be designed to emit lights of the same color or may be designed to emit lights of different colors. For example, in an inorganic LED having a laminated structure, the at least two inorganic LED light emitting units are favorably designed to emit lights of different colors to cause the inorganic LED to emit white light.

A type of inorganic LED called micro-LED is used in the form of an LED chip having a side of 100 μm or less. One side of a micro-LED chip is, for example, 5 μm or more and 100 μm or less, and the thickness thereof is, for example, 3 μm or more and 30 μm or less. Micro-LEDs are mounted on a substrate by a pick-and-place method. A pick-and-place method includes picking up the cut-out chips individually and placing them at the predetermined positions on a circuit board. With this method, a substrate can be assembled efficiently.

The light emitter 21 includes, sequentially from the supporting substrate 10 side, a reflection electrode 212 and a light emitting layer 213. In a plan view, at least part of the light emitting layer 213 overlaps the reflection electrode 212. This configuration allows light emitted from the light emitting layer 213 to be effectively reflected by the reflection electrode 212 to then be emitted toward the subject. As a result, the light sensitivity can be further enhanced.

The reflected light receiver 22 has a function of measuring the quantity of light emitted from the light emitter 21 and reflected in the living body of the subject (including light scattered in the living body). The reflected light receiver 22 is also referred to as a reflected (scattered) light receiving sensor.

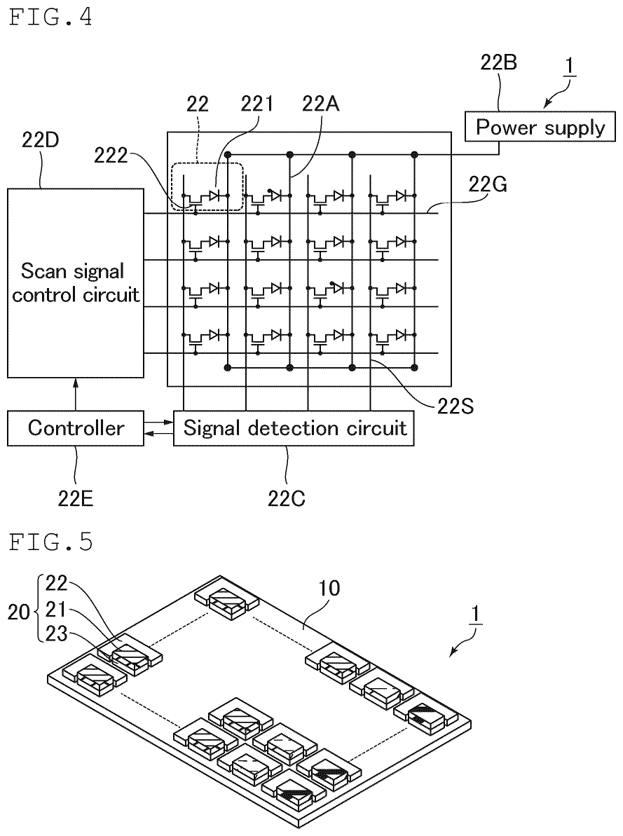

FIG. 4 is an example of a circuit diagram of a reflected light receiver in the wearable sensor of Embodiment 1. As shown in FIG. 3 and FIG. 4, the reflected light receiver 22 generates electric charges when receiving light, and includes a sensor unit 221 for reflected light reception used to accumulate the generated electric charges, and a switching element 222 for reflected light reception used to read out the electric charges accumulated in the sensor unit 221 for reflected light reception.

As shown in FIG. 4, the wearable sensor 1 includes, on the supporting substrate 10, gate lines 22G for reflected light reception parallel to one another and source lines 22S for reflected light reception parallel to one another and extending in a direction crossing the gate lines 22G for reflected light reception. The gate lines 22G for reflected light reception and the source lines 22S for reflected light reception as a whole are formed in a grid pattern to partition the sensor elements 20. Switching elements 222 for reflected light reception are placed at or near the respective intersections of the gate lines 22G for reflected light reception and the source lines 22S for reflected light reception. The gate lines 22G for reflected light reception are conductive lines used to turn on or off the switching elements 222 for reflected light reception. The source lines 22S for reflected light reception are conductive lines used to read out the electric charges accumulated in the sensor units 221 for reflected light reception.

As shown in FIG. 3, each reflected light receiver 22 includes, sequentially from the supporting substrate 10 side, a switching element 222 for reflected light reception and a sensor unit 221 for reflected light reception. Specifically, the reflected light receiver 22 includes, sequentially from the supporting substrate 10 side, a switching element 222 for reflected light reception, a first insulating film 241, a second insulating film 242, a sensor unit 221 for reflected light reception, a third insulating film 243, a fourth insulating film 244, and a fifth insulating film 245.

The sensor unit 221 for reflected light reception includes, sequentially from the supporting substrate 10 side, a bottom electrode 221A for reflected light reception, a photoelectric conversion layer 221B for reflected light reception, and a top electrode 221C for reflected light reception. The sensor unit 221 for reflected light reception includes, for example, an anode electrode and a cathode electrode (a bottom electrode 221A for reflected light reception and a top electrode 221C for reflected light reception), and a PIN diode (photoelectric conversion layer 221B for reflected light reception) sandwiched between the electrodes. The PIN diode is short for p-intrinsic-n diode and is a silicon diode including an I-type semiconductor, an intrinsic semiconductor with a large electrical resistance, between a P-type semiconductor and a N-type semiconductor which form a P-N junction.

The switching element 222 for reflected light reception includes, sequentially from the supporting substrate 10 side, a gate electrode 222G for reflected light reception, a gate insulating film 240, a semiconductor layer 222C for reflected light reception, a drain electrode 222D for reflected light reception, and a source electrode 222S for reflected light reception. The switching element 222 for reflected light reception may be, for example, a thin film transistor. The switching element 222 for reflected light reception preferably has a double-gate structure.

The semiconductor layer 222C for reflected light reception contains, for example, at least one of amorphous silicon, low-temperature polysilicon, or indium gallium zinc oxide. The semiconductor layer 222C for reflected light reception more preferably contains at least one of low-temperature polysilicon or indium gallium zinc oxide. In other words, the semiconductor layer 222C for reflected light reception more preferably contains LTPS or IGZO. This configuration can enhance the sensing characteristics. The semiconductor layer 222C for reflected light reception still more preferably contains IGZO with a low off-state leakage current.

The drain electrode 222D for reflected light reception is connected to the bottom electrode 221A for reflected light reception via a contact hole 22CH1. The source electrode 222S for reflected light reception is connected to the source line 22S for reflected light reception via a contact hole 22CH2.

In a plan view, at least part of the reflected light receiver 22 preferably does not overlap the light emitter 21. This configuration allows light reflected in the living body to be effectively received by the reflected light receiver 22, thus enhancing the light sensitivity.

Specifically, at least part of the photoelectric conversion layer 221B for reflected light reception in the reflected light receiver 22 preferably does not overlap the light emitting layer 213 in the light emitter 21. This configuration allows light reflected in the living body to be effectively received by the reflected light receiver 22, thus enhancing the light sensitivity.

In addition, the reflected light receiver 22 includes a common electrode line 22A for reflected light reception parallel to the source lines 22S for reflected light reception. The common electrode line 22A for reflected light reception has its ends connected in parallel, with one of the ends being connected to a power supply 22B for reflected light reception which supplies a predetermined bias voltage. The sensor unit 221 for reflected light reception is connected to the common electrode line 22A for reflected light reception, through which bias voltage is applied to the sensor unit 221 for reflected light reception.

Control signals for switching the switching elements 222 for reflected light reception flow through the gate lines 22G for reflected light reception. These control signals flowing through the gate lines 22G for reflected light reception cause the switching elements 222 for reflected light reception to be switched (turned on or off).

When the switching elements 222 for reflected light reception of the sensor elements 20 are in the on state, electrical signals corresponding to the electric charges accumulated in the respective sensor elements 20 flow to the corresponding source line 22S for reflected light reception. Specifically, when the switching element 222 for reflected light reception of any of the sensor element 20 connected to a source line 22S for reflected light reception is turned on, an electrical signal corresponding to the amount of electric charge accumulated in the sensor element 20 flows to the source line 22S for reflected light reception.

Each source line 22S for reflected light reception is connected to a signal detection circuit 22C for reflected light reception used to detect electrical signals flowing to the source lines 22S for reflected light reception. Each gate line 22G for reflected light reception is connected to a scan signal control circuit 22D for reflected light reception used to output control signals for turning on or off the switching elements 222 for reflected light reception to the gate lines 22G for reflected light reception. FIG. 4 shows one signal detection circuit 22C for reflected light reception and one scan signal control circuit 22D for reflected light reception, for simplification. Yet, for example, multiple signal detection circuits 22C for reflected light reception may be provided to each of which a predetermined number of source lines 22S for reflected light reception may be connected, and multiple scan signal control circuits 22D for reflected light reception may be provided to each of which a predetermined number of gate lines 22G for reflected light reception may be connected.

The signal detection circuit 22C for reflected light reception includes an internal amplifier circuit that amplifies input electrical signals for each source line 22S for reflected light reception. In the signal detection circuit 22C for reflected light reception, an electrical signal input from each source line 22S for reflected light reception is amplified by the amplifier circuit and is then converted to a digital signal by an analog-digital converter (ADC).

The signal detection circuit 22C for reflected light reception and the scan signal control circuit 22D for reflected light reception are connected to a controller 22E for reflected light reception which executes the predetermined processing including cancelling noise on digital signals converted by the signal detection circuit 22C for reflected light reception, outputs control signals indicating the timing of signal detection to the signal detection circuit 22C for reflected light reception, and outputs control signals indicating the timing of scan signal output to the scan signal control circuit 22D for reflected light reception.

The controller 22E for reflected light reception of the present embodiment is defined by a microcomputer and includes a non-volatile memory including a central processing unit (CPU), ROM and RAM, and a flash memory, for example. The controller 22E for reflected light reception generates an image indicated by the applied radiation based on the electrical signals input from the signal detection circuit 22C for reflected light reception, the signals carrying the electric charge information of the respective sensor elements 20.

The wearable sensor 1 of the present embodiment can be produced, for example, by the following production method. First, IGZO-TFTs are formed. The wearable sensor 1 requires TFTs for light emitting element control (switching elements 211 for light emission) and TFTs for in-vivo reflected light sensor control (switching elements 222 for reflected light reception). To enhance the controllability, the number of TFTs may be increased. IGZO may be replaced by LTSP or a-Si. In the case of IGZO-TFTs, a top-gate TFT structure or a double-gate TFT structure may also be employed.

Next, an anode electrode and a cathode electrode for light receiving sensor (reflected light receiver 22) (bottom electrode 221A for reflected light reception and top electrode 221C for reflected light reception) and a PIN diode (photoelectric conversion layer 221B for reflected light reception) to be sandwiched between them are formed. Then, the reflection electrode 212 for light emitting element (light emitter 21) and the light emitting layer 213 are formed.

The wearable sensor 1 includes, for each element (each sensor element 20), a light emitting element (light emitter 21) using an OLED display technology and a light receiving element (reflected light receiver 22) using an X-ray sensor technology. The sensor elements 20 are arranged in arrays having a certain area with high precision. The sensing quantities of all the sensor elements 20 are summed up to enable efficient sensing of light scattered in the living body.

### Embodiment 2

In the present embodiment, features unique to the present embodiment are mainly described and description of the same features as in Embodiment 1 is omitted. The present embodiment is substantially the same as Embodiment 1, except that the wearable sensor 1 includes emitted light receivers as well as the reflected light receivers 22.

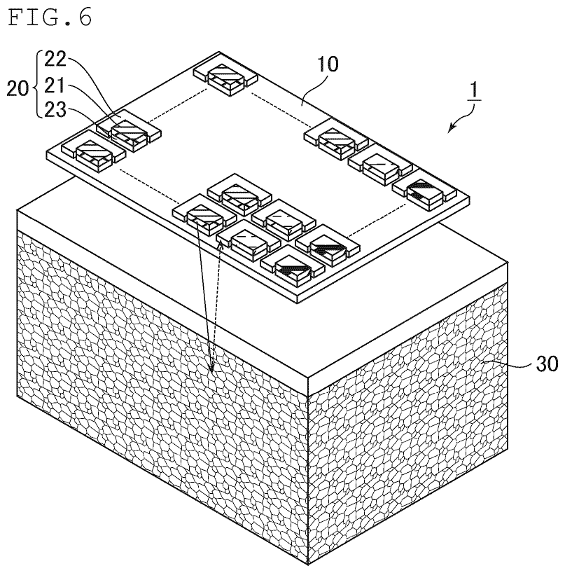

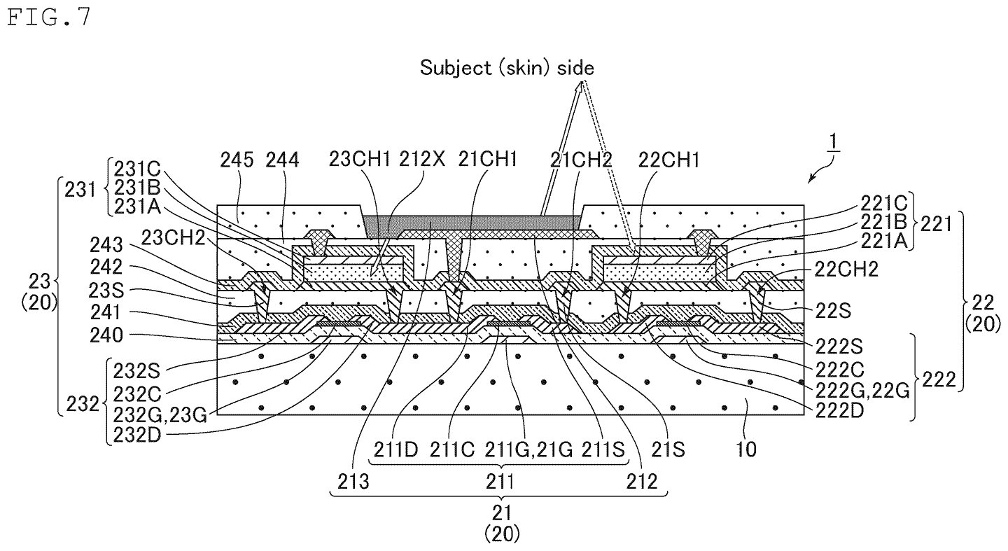

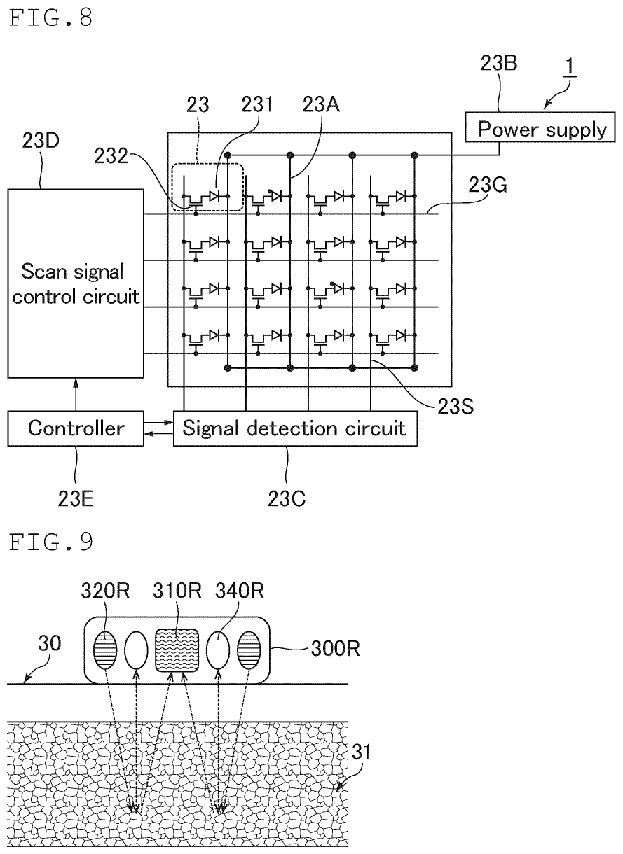

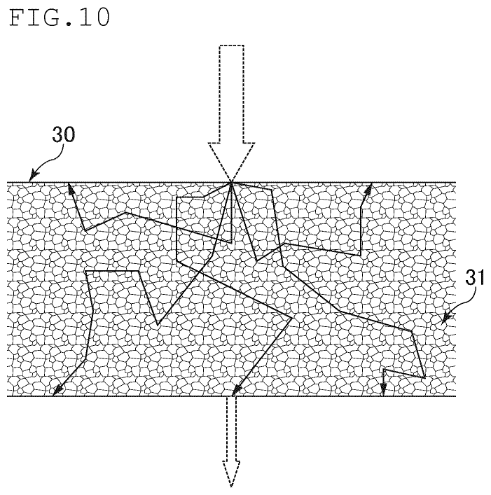

FIG. 5 is a schematic perspective view of a wearable sensor of Embodiment 2. FIG. 6 is a schematic view showing an example of a function of the wearable sensor of Embodiment 2. FIG. 7 is a schematic cross-sectional view of the wearable sensor of Embodiment 2. FIG. 8 is an example of a circuit diagram of an emitted light receiver in the wearable sensor of Embodiment 2. In each of FIG. 6 and FIG. 7, the solid line arrow indicates emission light from a light emitter, and the dashed line arrow indicates light having passed through a living body.

As shown in FIG. 5 to FIG. 8, the wearable sensor 1 of the present embodiment further includes emitted light receivers 23 each of which measures the quantity of light emitted from the light emitter 21 and not passing through the living body of a subject. The key to implement a biosensor is to detect the fluctuation of differences between the quantity of light emitted from the light emitting element (light emitter 21) and the quantity of light reflected in the living body. Thus, the wearable sensor 1 of the present embodiment includes, for each sensor element 20, a sensor (emitted light receiver 23) for measurement of quantity of light from the light emitting element (light emitter 21) as well as a sensor (reflected light receiver 22) for reception of light reflected in the living body. This configuration allows changes in a living body to be more accurately detected.

The wearable sensor 1 of the present embodiment specifically includes arrays of light emitting elements using an OLED display technology which correspond to the light emitters 21 and of semiconductor sensors using an X-ray sensor technology which correspond to the reflected light receivers 22 and the emitted light receivers 23 to enable a biosensor and thus solve the problem. Including high-definition light emitting elements and light receiving elements arranged in arrays having a certain area, the wearable sensor 1 can efficiently receive light reflected in a living body. The wearable sensor 1 is also less susceptible to the influence of differences in blood vessel distributions among individuals, thus having higher accuracy and higher sensitivity as a biosensor. In addition, including light emitting elements and semiconductor sensors formed on a flexible substrate, the wearable sensor 1 can be a device with improved wearability.

The emitted light receiver 23 has a function of measuring the quantity of light emitted from the light emitter 21 and not passing through the living body of a subject. The emitted light receiver 23 is also referred to as an emitted light receiving sensor.

As shown in FIG. 7 and FIG. 8, the emitted light receiver 23 generates electric charges when receiving light, and includes a sensor unit 231 for emitted light reception used to accumulate the generated electric charges, and a switching element 232 for emitted light reception used to read out the electric charges accumulated in the sensor unit 231 for reflected light reception.

As shown in FIG. 8, the wearable sensor 1 includes, on the supporting substrate 10, gate lines 23G for emitted light reception parallel to one another and source lines 23S for emitted light reception parallel to one another and extending in a direction crossing the gate lines 23G for emitted light reception. The gate lines 23G for emitted light reception and the source lines 23S for emitted light reception as a whole are formed in a grid pattern to partition the sensor elements 20. Switching elements 232 for emitted light reception are placed at or near the respective gate lines 23G for emitted light reception and the source lines 23S for emitted light reception. The gate lines 23G for emitted light reception are conductive lines used to turn on or off the switching elements 232 for emitted light reception. The source lines 23S for emitted light reception are conductive lines used to read out the electric charges accumulated in the sensor units 231 for emitted light reception.

As shown in FIG. 7, each emitted light receiver 23 includes, sequentially from the supporting substrate 10 side, a switching element 232 for emitted light reception and a sensor unit 231 for emitted light reception.

Specifically, the emitted light receiver 23 includes, sequentially from the supporting substrate 10 side, a switching element 232 for emitted light reception, a first insulating film 241, a second insulating film 242, a sensor unit 231 for emitted light reception, a third insulating film 243, a fourth insulating film 244, and a fifth insulating film 245.

The sensor unit 231 for emitted light reception includes, sequentially from the supporting substrate 10 side, a bottom electrode 231A for emitted light reception, a photoelectric conversion layer 231B for emitted light reception, and a top electrode 231C for emitted light reception. The sensor unit 231 for emitted light reception includes, for example, an anode electrode and a cathode electrode (a bottom electrode 231A for emitted light reception and a top electrode 231C for emitted light reception), and a PIN diode (photoelectric conversion layer 231B for emitted light reception) sandwiched between the electrodes. The PIN diode is short for p-intrinsic-n diode and is a silicon diode including an I-type semiconductor, an intrinsic semiconductor with a large electrical resistance, between a P-type semiconductor and a N-type semiconductor which form a P-N junction.

The switching element 232 for emitted light reception includes, sequentially from the supporting substrate 10 side, a gate electrode 232G for emitted light reception, a gate insulating film 240, a semiconductor layer 232C for emitted light reception, a drain electrode 232D for emitted light reception, and a source electrode 232S for emitted light reception. The switching element 232 for emitted light reception may be, for example, a thin film transistor. The switching element 232 for emitted light reception preferably has a double-gate structure.

The semiconductor layer 232C for emitted light reception contains, for example, at least one of amorphous silicon, low-temperature polysilicon, or indium gallium zinc oxide. The semiconductor layer 232C for emitted light reception more preferably contains at least one of low-temperature polysilicon or indium gallium zinc oxide. In other words, the semiconductor layer 232C for emitted light reception more preferably contains LTPS or IGZO. This configuration can enhance the sensing characteristics. The semiconductor layer 232C for emitted light reception still more preferably contains IGZO with a low off-state leakage current.

The drain electrode 232D for emitted light reception is connected to the bottom electrode 231A for emitted light reception via a contact hole 23CH1. The source electrode 232S for emitted light reception is connected to the source line 23S for emitted light reception via a contact hole 23CH2.

In a plan view, at least part of the emitted light receiver 23 preferably overlaps the light emitter 21. This configuration allows the emitted light receiver 23 to receive light emitted from the light emitter 21 more efficiently and allows the wearable sensor 1 to more accurately detect changes in the living body through comparison of the measurement results from the emitted light receiver 23 to those from the reflected light receiver 22.

Specifically, at least part of the photoelectric conversion layer 231B for emitted light reception in the emitted light receiver 23 preferably overlaps the light emitting layer 213 in the light emitter 21. This configuration allows the emitted light receiver 23 to receive light emitted from the light emitter 21 more effectively, thus allowing the wearable sensor 1 to more accurately detect changes in the living body.

Preferably, the light emitter 21 includes, sequentially from the supporting substrate 10 side, a reflection electrode 212 and a light emitting layer 213, the emitted light receiver 23 includes a photoelectric conversion layer 231B for emitted light reception as a light receiving element, the wearable sensor 1 includes, sequentially from the supporting substrate 10 side, the photoelectric conversion layer 231B for emitted light reception, the reflection electrode 212, and the light emitting layer 213, and in a plan view, at least part of the light emitting layer 213 overlaps the photoelectric conversion layer 231B for emitted light reception without the reflection electrode 212 in between. This configuration allows the quantity of light emitted from the light emitter 21 to be more accurately measured.

In a plan view, preferably, 1% or more and 20% or less of the area of the light emitting layer 213 overlaps the photoelectric conversion layer 231B for emitted light reception without the reflection electrode 212 in between.

Preferably, the light emitter 21 includes, sequentially from the supporting substrate 10 side, a reflection electrode 212 and a light emitting layer 213, the emitted light receiver 23 includes a photoelectric conversion layer 231B for emitted light reception as a light receiving element, the wearable sensor 1 includes, sequentially from the supporting substrate 10 side, the photoelectric conversion layer 231B for emitted light reception, the reflection electrode 212, and the light emitting layer 213, and in a plain view, at least part of a region of the reflection electrode 212 overlapping the photoelectric conversion layer 231B for emitted light reception is provided with an aperture 212X. This configuration allows the quantity of light emitted from the light emitter 21 to be more accurately measured.

In addition, the emitted light receiver 23 includes a common electrode line 23A for emitted light reception parallel to the source lines 23S for emitted light reception. The common electrode line 23A for emitted light reception has its ends connected in parallel, with one of the ends being connected to a power supply 23B for emitted light reception which supplies a predetermined bias voltage. The sensor unit 231 for emitted light reception is connected to the common electrode line 23A for emitted light reception, through which bias voltage is applied to the sensor unit 231 for emitted light reception.

Control signals for switching the switching elements 232 for emitted light reception flow through the gate lines 23G for emitted light reception. These control signals flowing through the gate lines 23G for emitted light reception cause the switching elements 232 for emitted light reception to be switched (turned on or off).

When the switching elements 232 for emitted light reception of the sensor elements 20 are in the on state, electrical signals corresponding to the electric charges accumulated in the respective sensor elements 20 flow to the corresponding source line 23S for emitted light reception. Specifically, when the switching element 232 for emitted light reception of any of the sensor elements 20 connected to a source line 23S for emitted light reception is turned on, an electrical signal corresponding to the amount of electric charge accumulated in the sensor element 20 flows to the source line 23S for emitted light reception.

Each source line 23S for emitted light reception is connected to a signal detection circuit 23C for emitted light reception used to detect electrical signals flowing to the source lines 23S for emitted light reception. Each gate line 23G for emitted light reception is connected to a scan signal control circuit 23D for emitted light reception used to output control signals for turning on or off the switching elements 232 for emitted light reception to the gate lines 23G for emitted light reception. FIG. 8 shows one signal detection circuit 23C for emitted light reception and one scan signal control circuit 23D for emitted light reception, for simplification. Yet, for example, multiple signal detection circuits 23C for emitted light reception may be provided to each of which a predetermined number of source lines 23S for emitted light reception may be connected, and multiple scan signal control circuits 23D for emitted light reception may be provided to each of which a predetermined number of gate lines 23G for emitted light reception may be connected.

The signal detection circuit 23C for emitted light reception includes an internal amplifier circuit that amplifies input electrical signals for each source line 23S for emitted light reception. In the signal detection circuit 23C for emitted light reception, an electrical signal input from each source line 23S for emitted light reception is amplified by the amplifier circuit and is then converted to a digital signal by an analog-digital converter (ADC).

The signal detection circuit 23C for emitted light reception and the scan signal control circuit 23D for emitted light reception are connected to a controller 23E for emitted light reception which executes the predetermined processing including cancelling noise on digital signals converted by the signal detection circuit 23C for emitted light reception, outputs control signals indicating the timing of signal detection to the signal detection circuit 23C for emitted light reception, and outputs control signals indicating the timing of scan signal output to the scan signal control circuit 23D for emitted light reception.

The controller 23E for emitted light reception of the present embodiment is defined by a microcomputer and includes a non-volatile memory including a central processing unit (CPU), ROM and RAM, and a flash memory, for example. The controller 23E for emitted light reception generates an image indicated by the applied radiation based on the electrical signals input from the signal detection circuit 23C for emitted light reception, the signals carrying the electric charge information of the respective sensor elements 20.

The wearable sensor 1 of the present embodiment can be produced, for example, by the following production method. First, IGZO-TFTs are formed. The wearable sensor 1 requires at least the following three TFTs: a TFT for light emitting element control (switching element 211 for light emission); a TFT for in-vivo reflected light sensor control (switching element 222 for reflected light reception); and a TFT for emitted light reception sensor control (switching element 232 for emitted light reception). To enhance the controllability, the number of TFTs may be increased. IGZO may be replaced by LTSP or a-Si. In the case of IGZO-TFTs, a top-gate TFT structure or a double-gate TFT structure may also be employed.

Next, an anode electrode and a cathode electrode for light receiving sensor (reflected light receiver 22 and emitted light receiver) (bottom electrode 221A for reflected light reception and top electrode 221C for reflected light reception, and bottom electrode 231A for emitted light reception and top electrode 231C for emitted light reception), and a PIN diode (photoelectric conversion layer 221B for reflected light reception and photoelectric conversion layer 231B for emitted light reception) to be sandwiched between them are formed. Then, the reflection electrode 212 for relight emitting element (light emitter 21) and the light emitting layer 213 are formed. At this time, to allow light to pass through the light emitting layer 213 to directly below the light emitting layer 213, the aperture 212X is formed in the reflection electrode 212. To make the quantity of light emission sufficient in this part, a transparent electrode may be formed in the aperture 212X.

The wearable sensor 1 includes, for each element (each sensor element 20), a light emitting element (light emitter 21) using an OLED display technology and a light receiving element (reflected light receiver 22 and emitted light receiver 23) using an X-ray sensor technology. The sensor elements 20 are arranged in arrays having a certain area with high precision. The sensing quantities of all the sensor elements 20 are summed up to enable efficient sensing of light scattered in the living body.

The wearable sensor 1 includes a light emitting element (light emitter 21) using an OLED display technology for each element (each sensor element 20) and a light receiving element (reflected light receiver 22 and emitted light receiver 23) using an X-ray sensor technology. The sensor elements 20 are arranged in arrays having a certain area with high precision. The sensing quantities of all the sensor elements 20 are summed up to enable efficient sensing of light scattered in the living body.

The wearable sensor 1 of the present embodiment includes two types of sensors, namely a sensor (emitted light receiver 23) used to measure the quantity of light from each light emitting element (light emitter 21) and a sensor (reflected light receiver 22) used to measure the reflected light in the living body. The fluctuation of differences in quantity of light between the two types of sensors is monitored, so that changes in the living body can be detected. The sensing quantities of all the sensor elements 20 are summed up to enable efficient sensing of light scattered in the living body. In addition, this configuration eliminates the need for control (unevenness reduction) of the quantity of light from each light emitting element, which is required for an OLED display, so that the number of TFT elements can be reduced and the unevenness reduction treatment is not required. Also, even when an OLED light emitting element deteriorates, degradation in light sensitivity can be reduced.

### EXAMPLES

Hereinafter, the effect of the present invention is described based on examples. The present invention is not limited to these examples.

### Example 1

A wearable sensor 1 of the present example corresponds to the wearable sensor 1 of Embodiment 1. The wearable sensor 1 of the present example is to be worn by a subject and includes a supporting substrate 10 and sensor elements 20 placed on the supporting substrate 10. The sensor elements 20 each include a light emitter 21 which is an OLED or an inorganic LED, and a reflected light receiver 22 which measures the quantity of light emitted from the light emitter 21 and reflected in the living body of a subject. The wearable sensor 1 of the present example can exhibit enhanced light sensitivity.

### Example 2

A wearable sensor 1 of the present example corresponds to the wearable sensor 1 of Embodiment 2. The wearable sensor 1 of the present example includes the structure in Embodiment 1 and further includes an emitted light receiver 23 which measures the quantity of light emitted from the light emitter 21 and not passing through the living body of a subject. The wearable sensor 1 of the present example can exhibit enhanced light sensitivity and can more accurately detect changes in the living body.

The embodiments of the present invention described above may be combined as appropriate without departing from the gist of the present invention.

What is claimed is:

1. A wearable sensor to be worn by a subject, the wearable sensor comprising:
a supporting substrate; and
sensor elements placed on the supporting substrate,
the sensor elements each comprising:
a light emitter which is an organic light emitting diode or an inorganic light emitting diode; and
a reflected light receiver configured to measure a quantity of light emitted from the light emitter and reflected in a living body of a subject.

2. The wearable sensor according to claim 1,
wherein in a plan view, at least part of the reflected light receiver does not overlap the light emitter.

3. The wearable sensor according to claim 1,
wherein the reflected light receiver comprises, sequentially from a supporting substrate side, a switching element for reflected light reception and a sensor unit for reflected light reception, and
the sensor unit for reflected light reception comprises, sequentially from the supporting substrate side, a bottom electrode for reflected light reception, a photoelectric conversion layer for reflected light reception, and a top electrode for reflected light reception.

4. The wearable sensor according to claim 3,
wherein the switching element for reflected light reception comprises a semiconductor layer for reflected light reception which contains at least one of amorphous silicon, low-temperature polysilicon, or indium gallium zinc oxide.

5. The wearable sensor according to claim 3,
wherein the switching element for reflected light reception has a double-gate structure.

6. The wearable sensor according to claim 1, further comprising an emitted light receiver configured to measure a quantity of light emitted from the light emitter and not passing through a living body of a subject.

7. The wearable sensor according to claim 6,
wherein the emitted light receiver comprises, sequentially from a supporting substrate side, a switching element for emitted light reception and a sensor unit for emitted light reception, and
the sensor unit for emitted light reception comprises, sequentially from the supporting substrate side, a bottom electrode for emitted light reception, a photoelectric conversion layer for emitted light reception, and a top electrode for emitted light reception.

8. The wearable sensor according to claim 7,
wherein the switching element for emitted light reception comprises a semiconductor layer for emitted light reception which contains at least one of amorphous silicon, low-temperature polysilicon, or indium gallium zinc oxide.

9. The wearable sensor according to claim 7,
wherein the switching element for emitted light reception has a double-gate structure.

10. The wearable sensor according to claim 6,
wherein the light emitter comprises, sequentially from a supporting substrate side, a reflection electrode and a light emitting layer,
the emitted light receiver comprises a photoelectric conversion layer for emitted light reception,
the wearable sensor comprises, sequentially from the supporting substrate side, the photoelectric conversion layer for emitted light reception, the reflection electrode, and the light emitting layer, and
in a plan view, at least part of the light emitting layer overlaps the photoelectric conversion layer for emitted light reception without the reflection electrode in between.

11. The wearable sensor according to claim 6,
wherein the light emitter comprises, sequentially from a supporting substrate side, a reflection electrode and a light emitting layer,
the emitted light receiver comprises a photoelectric conversion layer for emitted light reception,
the wearable sensor comprises, sequentially from the supporting substrate side, the photoelectric conversion layer for emitted light reception, the reflection electrode, and the light emitting layer, and
in a plan view, at least part of a region of the reflection electrode overlapping the photoelectric conversion layer for emitted light reception is provided with an aperture.

12. The wearable sensor according to claim 1,
wherein the light emitter comprises, sequentially from the supporting substrate side, a reflection electrode and a light emitting layer, and
in a plan view, at least part of the light emitting layer overlaps the reflection electrode.

13. The wearable sensor according to claim 1,
wherein the supporting substrate is a flexible substrate.
